(19) United States
(12) Patent Application Publication
(10) Pub. No.: US 2025/0332356 A1
(43) Pub. Date: Oct. 30, 2025

(54) Locking Connection for Syringe Adapter

(71) Applicant: Becton, Dickinson and Company, Franklin Lakes, NJ (US)

(72) Inventors: Pankaj Madhukar Patil, San Marcos, CA (US); Narsi Reddy Sanikommu, Kanigiri (IN); Ankita Kishor Jawale, Nashik (IN); Akshay Vidap, Solapur (IN); Nakul Sudarshan, Bengaluru (IN); Akash Kaloji, Pune (IN); Satyabrata Dalai, Khurda (IN); Mohammed Mehtab Khan, Bangalore (IN); Shwetha Mahadevappa, Tempe, AZ (US)

(21) Appl. No.: 18/646,159

(22) Filed: Apr. 25, 2024

(51) Int. Cl. A61M 5/34; A61J 1/20

(52) U.S. Cl. CPC A61M 5/345; A61J 1/2048

(57) ABSTRACT

A system for closed transfer of fluids includes a syringe adapter having a housing, a membrane at least partially received within an interior space of the housing, a needle hub positioned, a needle connected to the needle hub, a first connection interface defined by a portion of the housing, a connection release member moveable relative to the housing between a first position and a second position, and a secondary component including a component housing, a component membrane at least partially received within a passageway of the component housing, and a second connection interface positioned on the component housing. The second connection interface is configured to engage the first connection interface. The connection release member is configured to engage the second connection interface and release the second connection interface from the first connection interface when the connection release member is moved to the second position.

### BACKGROUND OF THE INVENTION

### Field of the Invention

The present invention relates to a locking connection for a syringe adapter.

### Description of Related Art

Health care providers reconstituting, transporting, and administering hazardous drugs, such as chemotherapeutic medication, can put health care providers at risk of exposure to these medications and present a hazard in the health care environment. Unintentional chemotherapy exposure can affect the nervous system, impair the reproductive system, and bring an increased risk of developing blood cancers in the future. Some drugs must be dissolved or diluted before they are administered, which involves transferring a solvent from one container to a sealed vial containing the drug in powder or liquid form. Drugs may be inadvertently released into the atmosphere in gas form or by way of aerosolization, during the withdrawal of the medication from the vial if any pressure differential between the interior of the vial and the surrounding atmosphere exists. In order to reduce the risk of health care providers being exposed to toxic drugs, the transfer of these drugs is accomplished utilizing a closed system transfer device or system.

Syringe adapters are utilized with a syringe to transfer medication from a container, such as a vial, to the syringe or to transfer a diluent from the syringe to the vial to reconstitute a medication. Syringe adapters are also utilized to transfer medication from the syringe to a patient's infusion line or to an infusion container, such as an IV bag, via an IV bag spike. Syringe adapters include a membrane received by a membrane housing, with the membrane of the syringe adapter configured to engage a corresponding membrane of a mating component, such as a patient connector, vial adapter, or IV bag spike, to ensure the closed transfer of fluid between the components such that the medication is not exposed to the ambient environment.

### SUMMARY OF THE INVENTION

In one aspect or embodiment, a system for closed transfer of fluids includes a syringe adapter having a housing including a first end and a second end positioned opposite the first end, with the housing includes a sidewall defining an interior space, a membrane at least partially received within the interior space of the housing and moveable from a first position within the housing to a second position within the housing spaced from the first position, a needle hub positioned at the first end of the housing and configured to be connected to a syringe, a needle connected to the needle hub, with the needle having a first end and a second end positioned opposite the first end, a first connection interface defined by a portion of the housing, and a connection release member moveable relative to the housing between a first position and a second position. The system also includes a secondary component including a component housing having a first end and a second end positioned opposite the first end, with the component housing includes a sidewall defining passageway, a component membrane at least partially received within the passageway, and a second connection interface positioned on the component housing, with the second connection interface configured to engage the first connection interface to secure the syringe adapter to the secondary component when the secondary component is at least partially received within the housing of the syringe adapter. The connection release member is configured to engage the second connection interface and release the second connection interface from the first connection interface when the connection release member is moved to the second position.

A portion of the component housing may be configured to be received within the interior space of the housing of the syringe adapter to move the membrane from the first position to the second position, where the first connection interface is configured to engage the second connection interface when the portion of the component housing is positioned within the interior space of the housing of the syringe adapter with the membrane in the second position. The syringe adapter may further include a membrane housing receiving the membrane, where the membrane housing is configured to connect to the component housing when the secondary component is at least partially received within the housing of the syringe adapter. The secondary component may include a luer connector. The syringe adapter may include a biasing member received within the housing, with the biasing member biasing the membrane toward the first position.

The first connection interface may be a ledge defined by the second end of the housing and the second connection interface may be a projection. The projection of the second connection interface may be positioned on a cantilever arm extending from the component housing, where engagement between the projection and the ledge prevents the secondary component from being removed from the syringe adapter. Engagement between the projection and the ledge may be a snap-fit and only disengaged via the connection release member. The connection release member may be an arm, where the arm is configured to be pressed by a healthcare to move a portion of the arm radially inward into engagement with the second connection interface.

In a further aspect or embodiment, a system for closed transfer of fluids includes a syringe adapter including a housing having a first end and a second end positioned opposite the first end, with the housing includes a sidewall defining an interior space, a membrane at least partially received within the interior space of the housing and moveable from a first position within the housing to a second position within the housing spaced from the first position, a needle hub positioned at the first end of the housing and configured to be connected to a syringe, and a needle connected to the needle hub, with the needle having a first end and a second end positioned opposite the first end. The system also includes a secondary component including a component housing having a first end and a second end positioned opposite the first end, with the component housing includes a sidewall defining passageway, and a component membrane at least partially received within the passageway. A locking collar is connected to and moveable relative to one of the syringe adapter and the secondary component, with the locking collar including a first threaded portion configured to engage a second threaded portion provided on the other of the syringe adapter and the secondary component when the secondary component is at least partially received within the housing of the syringe adapter.

The locking collar may be connected to the syringe adapter and moveable relative to the syringe adapter in axial and rotational directions, where the secondary component includes a flange including the second threaded portion. The syringe adapter may include a first connection interface defined by a portion of the housing, where the secondary component includes a second connection interface positioned on the component housing, with the second connection interface configured to engage the first connection interface to secure the syringe adapter to the secondary component when the secondary component is at least partially received within the housing of the syringe adapter. The locking collar may have a locked position where the first threaded portion is engaged with the second threaded portion when the first connection interface is engaged with the second connection interface and an unlocked position where the first threaded portion is disengaged from the second threaded portion. The syringe adapter may include a membrane housing receiving the membrane, where the membrane housing is configured to connect to the component housing when the secondary component is at least partially received within the housing of the syringe adapter. The secondary component may include a luer connector. The syringe adapter may include a biasing member received within the housing, with the biasing member biasing the membrane toward the first position.

The first connection interface may be a ledge defined by the second end of the housing and the second connection interface comprises a projection. The projection of the second connection interface may be positioned on a cantilever arm extending from the component housing, where engagement between the projection and the ledge connects the secondary component to the syringe adapter while allowing for disconnection of the secondary component upon separation of the secondary component from the syringe adapter. The locking collar may be connected to the secondary component and rotatable relative to the secondary component, where the second threaded portion is positioned on the housing of the syringe adapter.

In a further aspect or embodiment, a system for closed transfer of fluids includes a syringe adapter including a housing having a first end and a second end positioned opposite the first end, with the housing includes a sidewall defining an interior space, a membrane at least partially received within the interior space of the housing and moveable from a first position within the housing to a second position within the housing spaced from the first position, a needle hub positioned at the first end of the housing and configured to be connected to a syringe, and a needle connected to the needle hub, with the needle having a first end and a second end positioned opposite the first end. The system also includes a secondary component including a component housing having a first end and a second end positioned opposite the first end, with the component housing includes a sidewall defining passageway, and a component membrane at least partially received within the passageway. One of the housing of the syringe adapter and the component housing of the secondary component includes a latch arm, and the other of the housing of the syringe adapter and the component housing of the secondary component includes a latch post, with the latch arm configured to engage the latch post to secure the syringe adapter to the secondary component when the secondary component is at least partially received within the housing of the syringe adapter.

The latch arm may be positioned on the housing of the syringe adapter and moveable relative to the housing of the syringe adapter, where the latch post extends radially outward from the component housing of the secondary component.

In a further aspect or embodiments, a system for closed transfer of fluids includes a syringe adapter including a housing having a first end and a second end positioned opposite the first end, with the housing including an elliptical sidewall defining an interior space and the elliptical sidewall of the housing having a major axis and a minor axis, a membrane at least partially received within the interior space of the housing and moveable from a first position within the housing to a second position within the housing spaced from the first position, a needle hub positioned at the first end of the housing and configured to be connected to a syringe, and a needle connected to the needle hub, with the needle having a first end and a second end positioned opposite the first end. The system further includes a secondary component including a component housing having a first end and a second end positioned opposite the first end, with the component housing including an elliptical sidewall defining passageway and the elliptical sidewall of the component housing having a major axis and a minor axis, and a component membrane at least partially received within the passageway. With the major axis and the minor axis of the elliptical sidewall of the component housing aligned with the major axis and the minor axis of the elliptical sidewall of the housing, the secondary component is configured to be at least partially inserted into the housing of the syringe adapter. With the secondary component at least partially inserted into the housing of the syringe adapter, the secondary component is configured to be locked to the syringe adapter when the major axis of the elliptical sidewall of the component housing is aligned with the minor axis of the elliptical sidewall of the housing.

### BRIEF DESCRIPTION OF THE DRAWINGS

FIG. 1 is a front view of a syringe adapter and patient connector according to one aspect or embodiment of the present application, showing the syringe adapter and patient connector disconnected;

FIG. 2 is a cross-sectional view of the syringe adapter and patient connector of FIG. 1, showing the syringe adapter in the process of being connected to the patient connector;

FIG. 3 is a cross-sectional view of the syringe adapter and patient connector of FIG. 1, showing the syringe adapter in the process of being connected to the patient connector;

FIG. 4 is a cross-sectional view of the syringe adapter and patient connector of FIG. 1, showing the syringe adapter connected to the patient connector;

FIG. 5 is a partial cross-sectional view of the syringe adapter and patient connector of FIG. 1, showing the syringe adapter connected to the patient connector;

FIG. 6 is a partial front view of the syringe adapter and patient connector of FIG. 1, showing the syringe adapter connected to the patient connector with a portion of the housing of the syringe adapter transparent for clarity;

FIG. 7 is a partial cross-sectional view of the syringe adapter and patient connector of FIG. 1, showing the syringe adapter connected to the patient connector;

FIG. 8 is a partial cross-sectional view of the syringe adapter and patient connector of FIG. 1, showing the syringe adapter in the process of being disconnected to the patient connector;

FIG. 9 is a partial cross-sectional view of the syringe adapter and patient connector of FIG. 1, showing the syringe adapter in the process of being disconnected to the patient connector;

FIG. 10 is a perspective view of a conventional infusion clamp, showing the infusion clamp being connected to a syringe adapter;

FIG. 11 is a perspective view of a conventional infusion clamp, showing the infusion clamp connected to a syringe adapter;

FIG. 12 is a perspective view of a conventional infusion clamp, showing the infusion clamp being disconnected to a syringe adapter;

FIG. 13 is a cross-sectional view of a syringe adapter according to one aspect or embodiment of the present application;

FIG. 14 is a bottom perspective view of the syringe adapter of FIG. 13;

FIG. 15 is a perspective view of a patient connector according to one aspect or embodiment of the present application;

FIG. 16 is a cross-sectional view of the syringe adapter of FIG. 13 and the patient connector of FIG. 15, showing the syringe adapter connected to the patient connector;

FIG. 17 is a perspective view of the syringe adapter of FIG. 13 and the patient connector of FIG. 15, showing the syringe adapter being connected to the patient connector;

FIG. 18 is a front view of the syringe adapter of FIG. 13 and the patient connector of FIG. 15, showing the syringe adapter connected to the patient connector;

FIG. 19 is a cross-sectional view of the syringe adapter of FIG. 13 and the patient connector of FIG. 15, showing the syringe adapter connected to the patient connector;

FIG. 20 is a front view of the syringe adapter of FIG. 13 and the patient connector of FIG. 15, showing connection of a locking collar;

FIG. 21 is a front view of the syringe adapter of FIG. 13 and the patient connector of FIG. 15, showing disconnection of a locking collar;

FIG. 22 is a perspective view of the syringe adapter of FIG. 13 and the patient connector of FIG. 15, showing the syringe adapter disconnected from the patient connector;

FIG. 23 is a partial cross-sectional view of a syringe adapter and patient connector according to a further aspect or embodiment of the present application, showing the syringe adapter connected to the patient connector;

FIG. 24 is a partial cross-sectional view of a syringe adapter and patient connector according to a further aspect or embodiment of the present application, showing the syringe adapter connected to the patient connector;

FIG. 25 is a front view of a syringe adapter and patient connector according to a further aspect or embodiment of the present application, showing the syringe adapter connected to the patient connector;

FIG. 26 is a partial perspective view of the syringe adapter and patient connector of FIG. 25;

FIG. 27 is a front view of the syringe adapter and patient connector of FIG. 25, showing the syringe adapter being connected to the patient connector;

FIG. 28 is a front view of the syringe adapter and patient connector of FIG. 25, showing the syringe adapter connected to the patient connector;

FIG. 29 is a perspective view of a syringe adapter according to a further aspect or embodiment of the present application;

FIG. 30 is a bottom view of a patient connector according to a further aspect or embodiment of the present application;

FIG. 31 is a perspective view of the syringe adapter of FIG. 29 and the patient connector of FIG. 30, showing the syringe adapter connected to the patient connector;

FIG. 32 is a cross-sectional view of the syringe adapter of FIG. 29 and the patient connector of FIG. 30, showing the syringe adapter connected to the patient connector;

FIG. 33 is a cross-sectional view of the syringe adapter of FIG. 29 and the patient connector of FIG. 30, showing the syringe adapter connected to the patient connector;

FIG. 34 is a perspective view of the syringe adapter of FIG. 29 and the patient connector of FIG. 30, showing the syringe adapter connected to the patient connector with a secure connection;

FIG. 35 is a cross-sectional view of the syringe adapter of FIG. 29 and the patient connector of FIG. 30, showing the syringe adapter connected to the patient connector with a secure connection; and

FIG. 36 is a cross-sectional view of the syringe adapter of FIG. 29 and the patient connector of FIG. 30, showing the syringe adapter connected to the patient connector with a secure connection.

### DETAILED DESCRIPTION

For purposes of the description hereinafter, the terms such as “end”, “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting. Further, it is to be understood that the invention may assume various alternative variations and step sequences, except where expressly specified to the contrary.

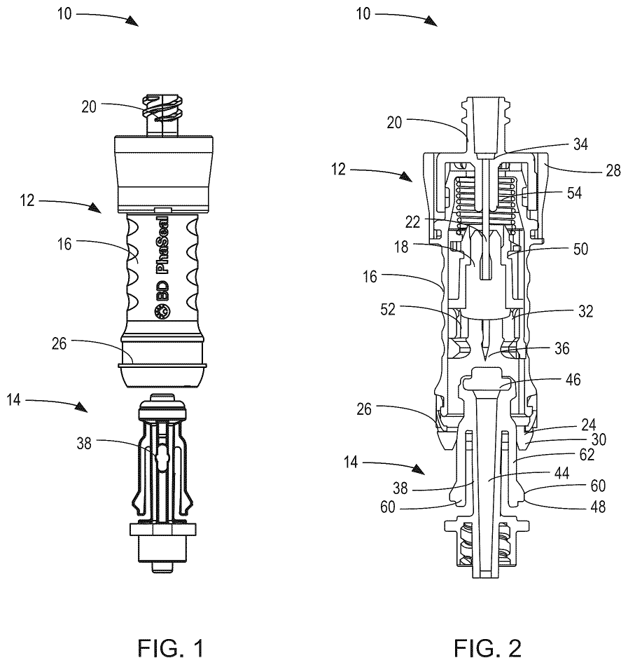

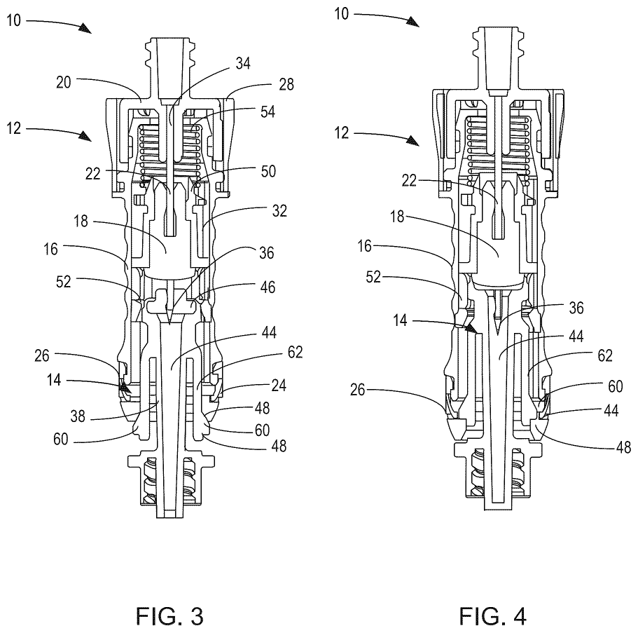

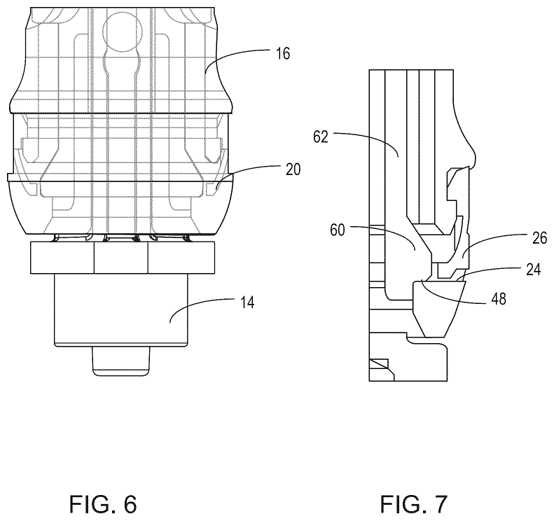

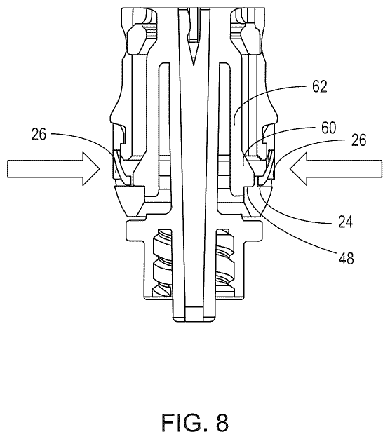

Referring to FIGS. 1-9, a system 10 for the closed transfer of fluids according to one aspect or embodiment of the present application is shown is shown. The system 10 includes a syringe adapter 12 and a secondary component 14, which are components of the system 10 for the closed transfer of fluids. The syringe adapter 12 is configured to connect to a syringe (not shown) and to the secondary component 14, which may facilitate connection to another medical device or fluid container. The medical device can be, for example, a patient line, vial adapter, fluid container, or infusion adapter. The fluid container can be a medical vial, syringe barrel, IV bag, or similar container for holding a fluid to be administered to a patient. The syringe adapter 12 can be used to facilitate the transfer of fluids between the syringe and secondary component 14 or fluid container while preventing dripping of the fluid or expelling of the fluid outside of the system. The syringe adapter 12 and secondary component 14 may be similar to and function similarly to the syringe adapter and patient connector shown and described in U.S. Patent Application Publication No. 2018/0200147, which is hereby incorporated by reference in its entirety. In some aspects or embodiments, the secondary component 14 is a patient connector, as shown in FIGS. 1-9.

Referring again to FIGS. 1-9, the syringe adapter 12 includes a housing 16, a membrane 18, a needle hub 20, a needle 22 connected to the needle hub 20, a first connection interface 24 defined by a portion of the housing 16, and a connection release member 26 moveable relative to the housing 16 between a first position and a second position. The housing 16 has a first end 28 and a second end 30 positioned opposite the first end 28, with the housing 16 including a sidewall defining an interior space 32. The membrane 18 is at least partially received within the interior space 32 of the housing 16 and moveable from a first position within the housing 16 to a second position within the housing 16 spaced from the first position. The needle hub 20 is positioned at the first end 28 of the housing 16 and is configured to be connected to a syringe. The needle 22 has a first end 34 and a second end 36 positioned opposite the first end 34. The secondary component 14 includes a component housing 38 having a first end 40 and a second end 42 positioned opposite the first end 40, with the component housing 38 includes a sidewall defining passageway 44, a component membrane 46 at least partially received within the passageway 44, and a second connection interface 48 positioned on the component housing 38. The second connection interface 48 is configured to engage the first connection interface 24 to secure the syringe adapter 12 to the secondary component 14 when the secondary component 14 is at least partially received within the housing 16 of the syringe adapter 12. The connection release member 26 is configured to engage the second connection interface 48 and release the second connection interface 48 from the first connection interface 24 when the connection release member 26 is moved to the second position.

Referring to FIGS. 2-4, a portion of the component housing 38 is configured to be received within the interior space 32 of the housing 16 of the syringe adapter 12 to move the membrane 18 from the first position to the second position. The first connection interface 24 is configured to engage the second connection interface 48 when the portion of the component housing 38 is positioned within the interior space 32 of the housing 16 of the syringe adapter 12 with the membrane 18 in the second position. The syringe adapter 12 includes a membrane housing 50 with a collet 52, with the membrane housing 50 receiving the membrane 18. The membrane housing 50 is configured to connect to the component housing 38 via the collet 52 when the secondary component 14 is at least partially received within the housing 16 of the syringe adapter 12. The needle hub 20 may include a luer connector, although other suitable connection arrangements may be utilized. The second end 36 of the needle 22 is positioned within the membrane 18 when the membrane 18 is in the first position and the second end 36 of the needle 22 is positioned outside of the membrane 18 when the membrane 18 is in the second position. In some aspects or embodiments, the needle hub 20 is rotatable relative to the housing 16, which inhibits accidental disconnection of a syringe from the needle hub 20 by requiring a healthcare worker to grasp the needle hub 20 to connect and/or disconnect a syringe from the needle hub 20. The secondary component 14 may include a luer connector. A biasing member 54, such as a compression spring, may be received within the housing 16 to bias the membrane 18 and the membrane housing 50 toward the first position.

Referring to FIGS. 1-4, when the secondary component 14 is inserted into the housing 16 of the syringe adapter 12, the collet 52 of the membrane housing 50 receives the secondary component 14 and moves the membrane housing 50 within the housing 16 toward the first end 28 of the housing 16. As the membrane housing 50 moves, the collet 52 secures the membrane housing 50 to the secondary component 14 to ensure engagement between the component membrane 46 and the membrane 18 of the membrane housing 50. As the membrane 18 and membrane housing 50 are moved from the first position to the second position, the second end 36 of the needle 22 pierces the membrane 18 of the membrane housing 50 and the component membrane 46 of the secondary component 14 such that the second end 36 is received within the passageway 44 of the secondary component 14 thereby placing the needle 22 in fluid communication with the secondary component 14. The needle hub 20 is in fluid communication with the needle 22 to allow a syringe (not shown) connected to the needle hub 20 to be in fluid communication with the secondary component 14 when the syringe adapter 12 is connected to the secondary component 14. Moving the membrane 18 and membrane housing 50 from the second position to the first position, which is aided by the biasing member 54, releases the secondary component 14 from the collet 52.

The membrane 18 and the membrane housing 50 are further secured in the second position by the second connection interface 48 engaging the first connection interface 24. In some aspects or embodiments, the first connection interface 24 is a ledge defined by the second end 30 of the housing 16 or another portion of the housing 16. The second connection interface 48 is a projection 60. In some aspects or embodiments, the projection 60 of the second connection interface 48 is positioned on a cantilever arm 62 extending from the component housing 38. Engagement between the projection 60 and the ledge prevents the secondary component 14 from being removed from the syringe adapter 12. In some aspects or embodiments, engagement between the projection 60 and the ledge is a snap-fit and can only be disengaged via the connection release member 26. In some aspects or embodiments, the connection release member 26 is an arm, where the arm is configured to be pressed by a healthcare to move a portion of the arm radially inward into engagement with the second connection interface 48, which, in turn, moves the cantilever arm 62 of the secondary component 14 radially inward and the projection 60 out of engagement with the ledge thereby allowing the biasing member 54 or other axial force to separate the secondary component 14 of the syringe adapter 12. Although two connection release members 26 are shown, one or more connection release members 26 may be provided. In some aspects or embodiments, due to the shape of the projection 60 and the cantilever arm 62, the secondary component 14 can be inserted into the housing of the syringe adapter 12 to deflect the cantilever arm 62 and allow connection of the secondary component 14 to the syringe adapter 12, but cannot be removed from the housing 16 of the syringe adapter 12 unless the connection release member 26 is pressed and moved radially inwardly by a healthcare worker. Accordingly, the syringe adapter 12 and the secondary component 14 are locked to each other until there is deliberate action by healthcare worker to separate the components by engaging the connection release member 26, which reduces the likelihood of accidental disconnection between the components of the system 10 and exposure of hazardous drugs.

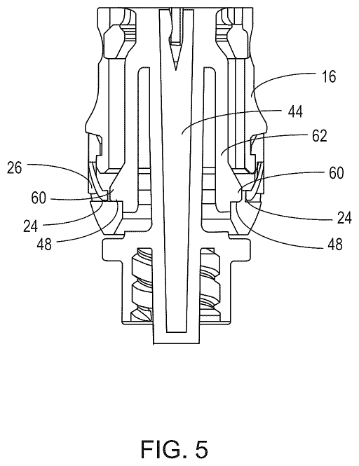

Referring to FIGS. 5-7, the connection between the projection 60 of the secondary component 14 and the first connection interface 24 or ledge of the syringe adapter 12 forms a 90 degree snap-fit connection. In other words, when the secondary component 14 is connected to the syringe adapter 12, a planar surface of the projection 60 is engaged with a planar surface of the first connection interface 24 or ledge such that applying an axial force to the secondary component 14 away from the syringe adapter 12 will not deflect the cantilever arm 62 radially inward to allow the connection between the first connection interface 24 and the projection 60 to be released. The component release member 26 may be connected to the housing 16 or formed integrally with the housing 16 and may be include a compliant connection to the housing 16, such as a living hinge or other suitable arrangement.

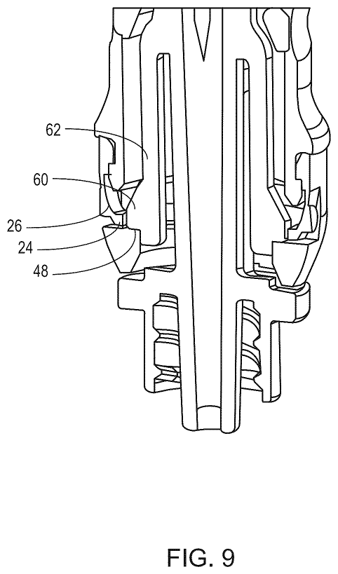

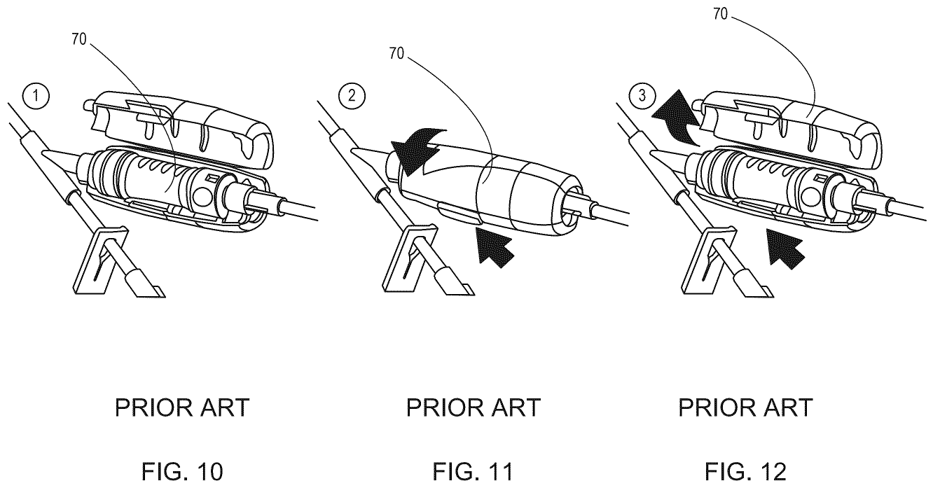

Referring to FIGS. 10-12, a conventional arrangement for securing a connection between a syringe adapter and secondary component includes an infusion clamp 70. After the syringe adapter is connected to the secondary component, the infusion clamp 70 is positioned over the syringe adapter and the secondary component (FIG. 10) and subsequently closed over connected components (FIG. 11) to prevent accidental disconnection. The infusion clamp 70 is opened and removed from the connected components (FIG. 12) to allow for disconnection of the components. The infusion clamp 70, however, is an additional component and adds complexity to the process and inventory management. An integrated solution for ensuring the connection between the components is desired.

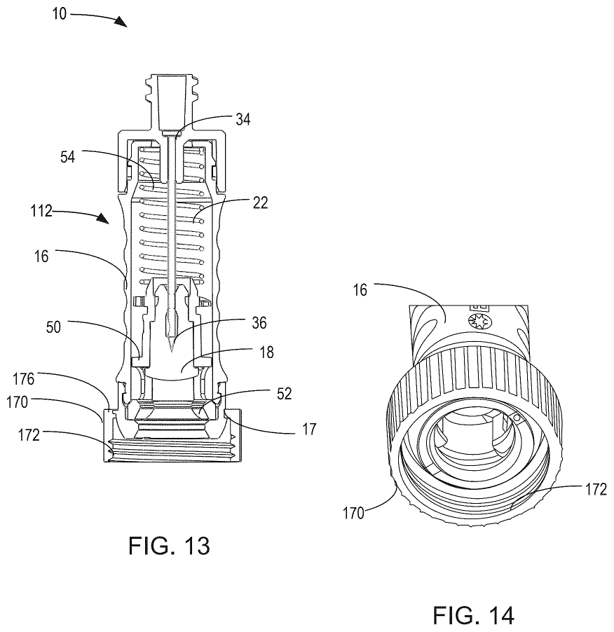

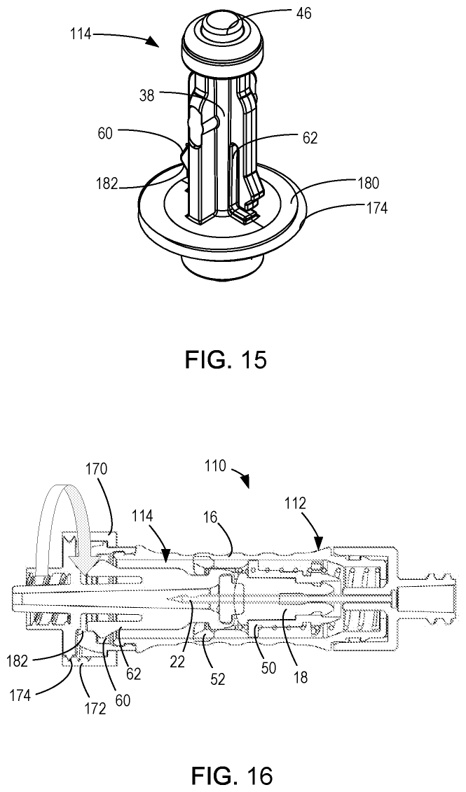

Referring to FIGS. 13-24, a system 110 for closed transfer of fluids according to a further aspect or embodiment of the present application is shown. The system includes a syringe adapter 112 and a secondary component 114, which are similar to and operate similarly to the syringe adapter 12 and the secondary component 14 shown in FIGS. 1-9 and like reference numbers are utilized for like components. The syringe adapter 112 and the secondary component 114 do not include the connection release member 26 and the projection 60 of the second connection interface 48 is different, as discussed in more detail below. The system 110 of FIGS. 13-24 includes a locking collar 170 connected to and moveable relative to one of the syringe adapter 112 and the secondary component 114. The locking collar 170 is configured to be rotated by a healthcare worker to secure a connection between the syringe adapter 112 and the secondary component 114. The locking collar 170 includes a first threaded portion 172 configured to engage a second threaded portion 174 provided on the other of the syringe adapter 112 and the secondary component 114 when the secondary component 114 is at least partially received within the housing 16 of the syringe adapter 112.

Referring FIGS. 13-22, in some aspects or embodiments, the locking collar 170 is connected to the syringe adapter 112 and moveable relative to the syringe adapter 112 in axial and rotational directions. The locking collar 170 is annular, although other suitable shapes may be utilized, and is positioned over the syringe adapter 112 with corresponding flanges 176, 178 of the locking collar 170 and the housing 16 of the syringe adapter 112 acting as an axial stop. The secondary component 114 has a flange 180 including the second threaded portion 174. The syringe adapter 112 and the secondary component 114 includes the first connection interface 24 and the second connection interface 48, as discussed above in connection with FIGS. 1-9, but the second connection interface 48 of the secondary component 114 includes an angled surface 182 that allows for removal of the secondary component 114 from the syringe adapter 112 by applying an axial force to the secondary component 114 that is sufficient to overcome the biasing force of the cantilever arm 62 due to the camming action between the housing 16 of the syringe adapter 112. The locking collar 170 has a locked position where the first threaded portion 172 is engaged with the second threaded portion 174 when the first connection interface 24 is engaged with the second connection interface 48 and an unlocked position where the first threaded portion 172 is disengaged from the second threaded portion 174. The first threaded portion 172 includes an internal helical thread and the second threaded portion includes an external helical thread configured to mate and engage the internal helical thread.

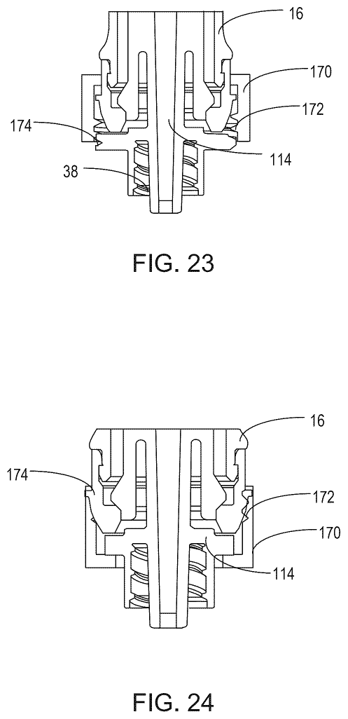

Referring to FIG. 23, in some aspects or embodiments, the first threaded portion 172 includes an external helical thread and the second threaded portion 174 includes an internal helical thread configured to mate and engage the external helical thread.

Referring to FIG. 24, in some aspects or embodiments, the locking collar 170 is connected to the secondary component 114 and rotatable relative to the secondary component 114 and the second threaded portion 174 is positioned on the housing 16 of the syringe adapter 112.

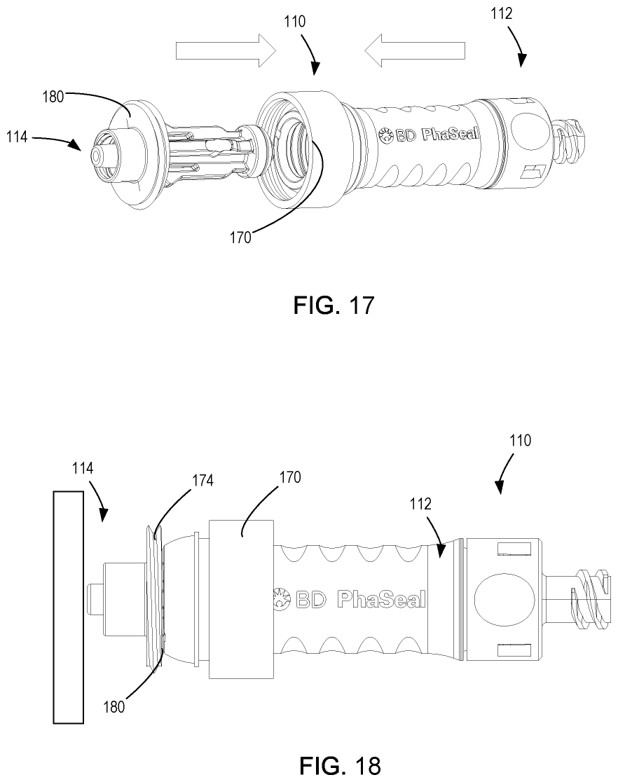

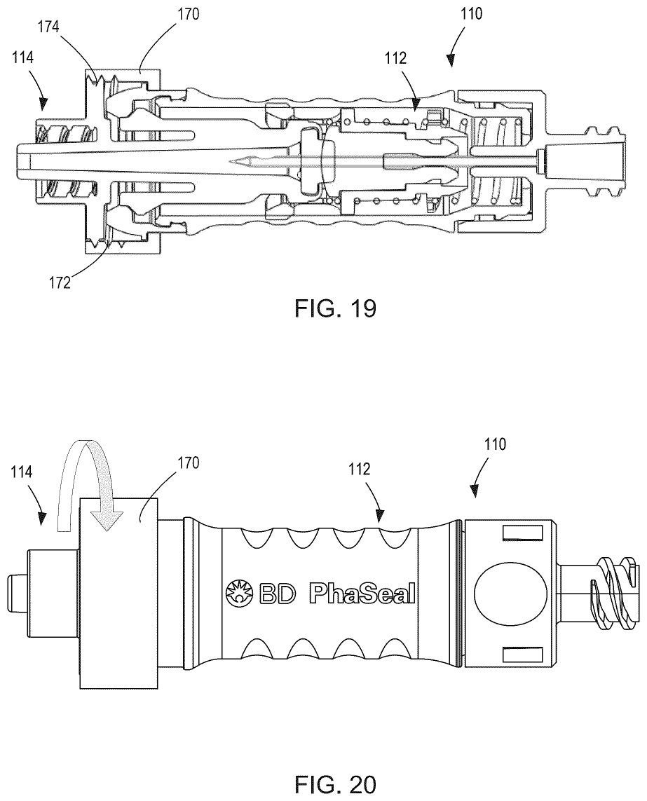

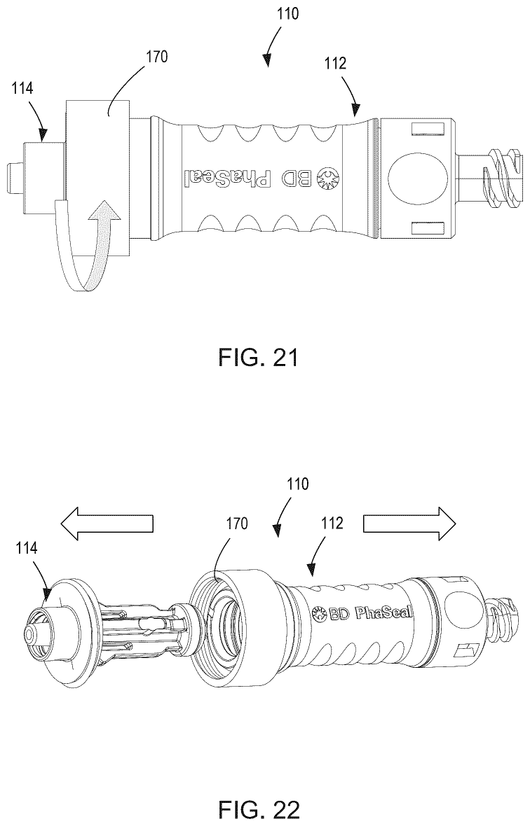

Referring to FIGS. 17-22, according to one aspect or embodiment, the system 110 is utilized by inserting the secondary component 114 into the syringe adapter 112 (FIGS. 17 and 18) with the collet 52 engaging the secondary component 114 and the first connection interface 24 engaging the second connection interface 48. The locking collar 170 is moved axially (FIGS. 19 and 20) until the first threaded portion 172 abut the second threaded portion 174 and then rotated to engage the first threaded portion 172 with the second threaded portion 174 thereby securing the secondary component 114 to the syringe adapter 112. The secondary component 114 can be separated from the syringe adapter 112 (FIGS. 21 and 22) by unthreading the locking collar 170 by rotating the locking collar 170 is an opposite direction and axially separated the secondary component 114 from the syringe adapter 112. The locking collar 170 can be slid or axially translated away from the second end 30 of the housing 16 if use of the locking collar 170 is not needed or desired.

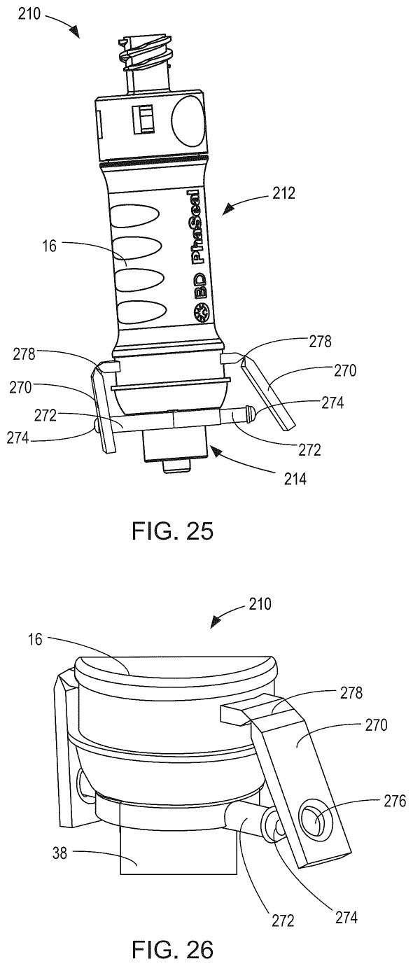

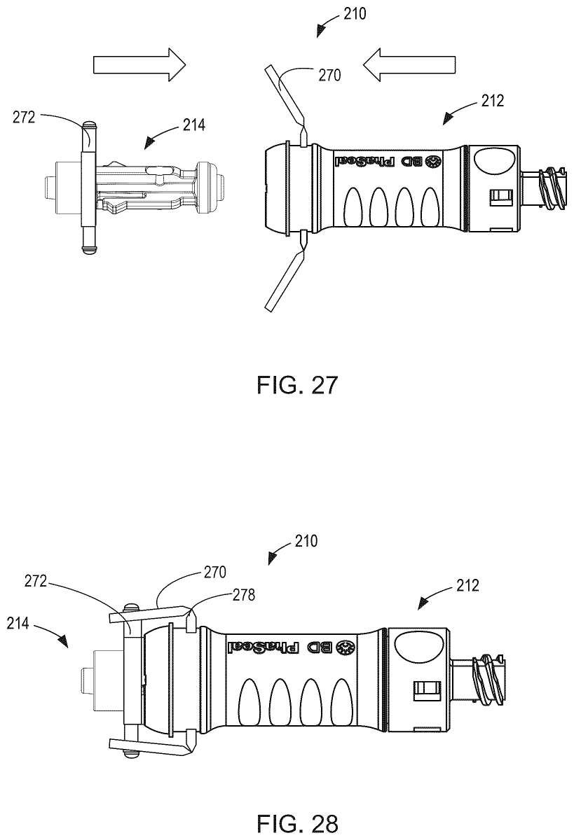

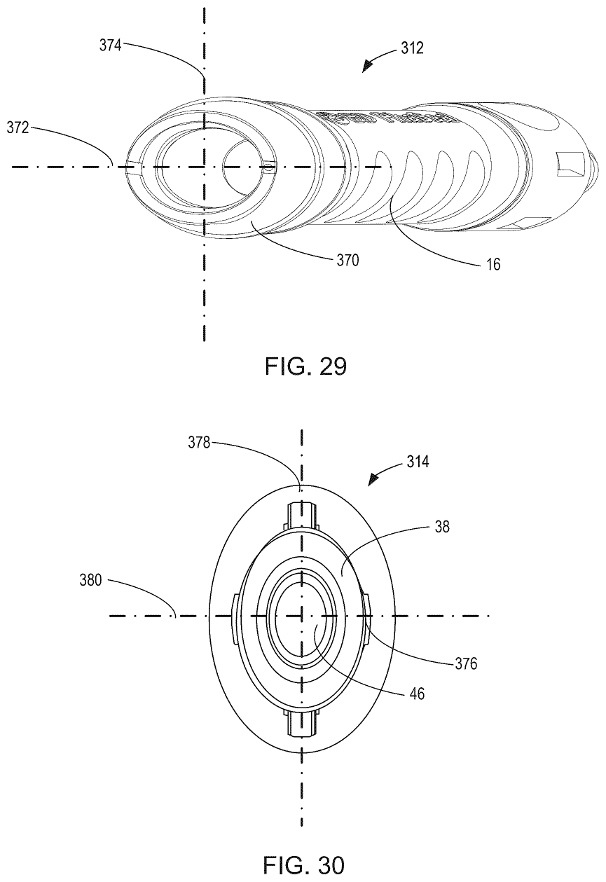

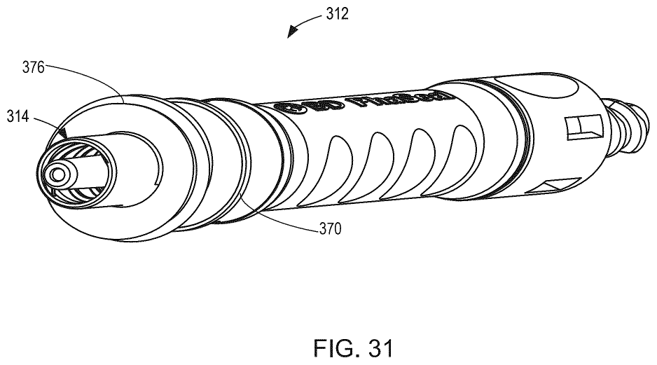

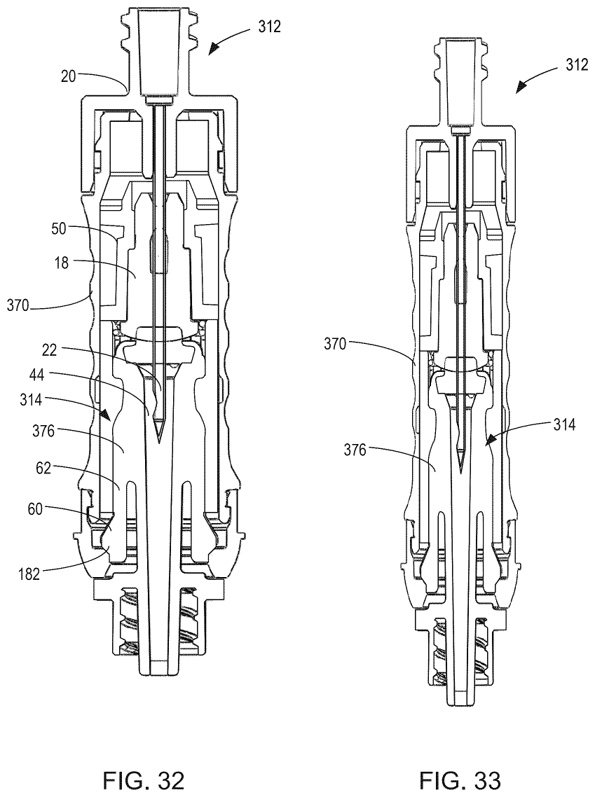

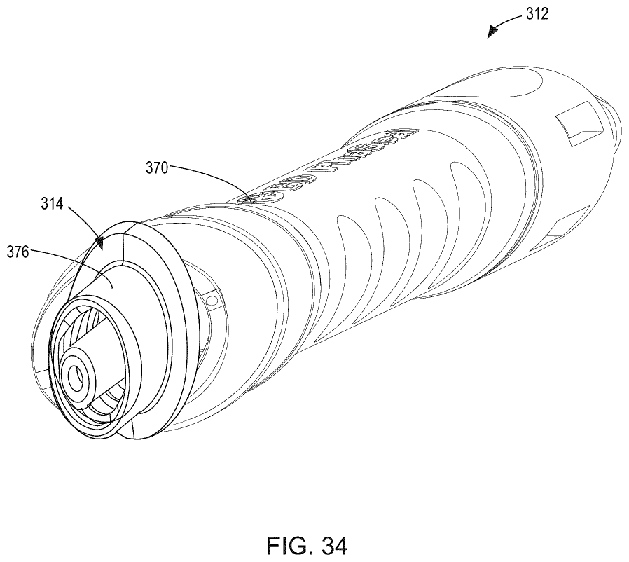

Referring to FIGS. 25-28, a system 210 for closed transfer of fluids according to a further aspect or embodiment of the present application is shown. The system 210 includes a syringe adapter 212 and a secondary component 214, which are similar to and operate similarly to the syringe adapter 12 and the secondary component 14 shown in FIGS. 1-9 and like reference numbers are utilized for like components. The syringe adapter 212 and the secondary component 214 do not include the connection release member 26 to ensure a secure connection. To ensure a secure connection between the syringe adapter 212 and the secondary component 214, one of the housing 16 of the syringe adapter 212 and the component housing 38 of the secondary component 214 includes a latch arm 170. The other of the housing 16 of the syringe adapter 212 and the component housing 38 of the secondary component 214 includes a latch post 272, with the latch arm 270 configured to engage the latch post 272 to secure the syringe adapter 212 to the secondary component 214 when the secondary component 214 is at least partially received within the housing 16 of the syringe adapter 212.

In some aspects or embodiments, as shown in FIGS. 25-28, the latch arm 270 is positioned on the housing 16 of the syringe adapter 212 and moveable relative to the housing 16 of the syringe adapter 212, with the latch post 272 extending radially outward from the component housing 38 of the secondary component 214. The latch post 272 is cylindrical and includes a head portion 274 to create a snap fit when the latch post 272 is received within an opening 276 of the latch arm 270, although other suitable arrangements may be utilized. In some aspects or embodiments, the latch arm 270 is rotatable relative to the housing 16 via a pivot point 278, which may be a living hinge or other suitable arrangement. Although two latch arms 270 are shown in FIGS. 25-28, one or more latch arms 270 and one or more latch posts 272 may be provided.

Referring to FIGS. 39-36, a system 310 for closed transfer of fluids according to a further aspect or embodiment of the present application is shown. The system 310 includes a syringe adapter 312 and a secondary component 314, which are similar to and operate similarly to the syringe adapter 12 and the secondary component 14 shown in FIGS. 1-9 and like reference numbers are utilized for like components. The syringe adapter 312 and the secondary component 314 do not include the connection release member 26 to ensure a secure connection. To ensure a secure connection between the syringe adapter 312 and the secondary component 314, the housing 16 of the syringe adapter 312 includes an elliptical sidewall 370 having a major axis 372 and a minor axis 374 and the component housing 38 of the secondary component 314 includes an elliptical sidewall 376 having a major axis 378 and a minor axis 380. With the major axis 378 and the minor axis 380 of the elliptical sidewall 376 of the component housing 38 aligned with the major axis 372 and the minor axis 374 of the elliptical sidewall 370 of the housing 16, the secondary component 314 is configured to be at least partially inserted into the housing 16 of the syringe adapter 312. With the secondary component 314 at least partially inserted into the housing 16 of the syringe adapter 312, the secondary component 314 is configured to be locked to the syringe adapter 312 when the major axis 378 of the elliptical sidewall 376 of the component housing 38 is aligned with the minor axis 374 of the elliptical sidewall 370 of the housing 16.

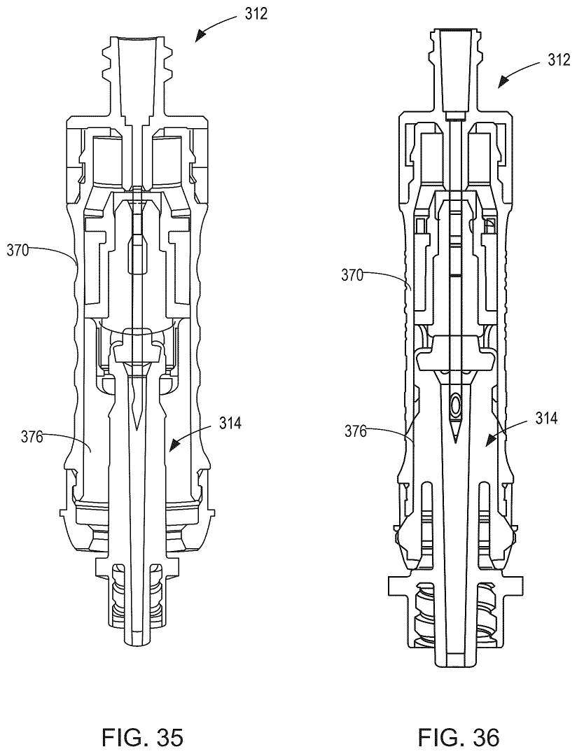

Referring to FIGS. 35 and 36, in some aspects or embodiments, the secondary component 314 is configured to be locked to the syringe adapter 312 when the major axis 378 of the elliptical sidewall 376 of the component housing 38 is aligned with the minor axis 374 of the elliptical sidewall 370 of the housing 16 by rotating the secondary component 314 ninety degrees relative to the syringe adapter 312, which compresses the connection between the collet 52 and the secondary component 314 and/or compresses the first connection interface 24 with the second connection interface 48.

The systems 10, 110, 210, 310 discussed above and the related features for ensuring a secure connection between the syringe adapter and the secondary component eliminates the need for additional components, such as the infusion clamp 70, reduces material costs, improves the workflow process by not requiring an additional component to ensure a secure connection, reduces the chance of misplacing an additional component once the additional component is removed, provides for a compact design, and reduces packaging cost and waste, while still ensuring a secure connection between the components.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims. To the extent possible, one or more features of any aspect or embodiment discussed above can be combined with one or more features of any other aspect or embodiment.

The invention claimed is:

1. A system for closed transfer of fluids comprising:
a syringe adapter comprising:
a housing having a first end and a second end positioned opposite the first end, the housing includes a sidewall defining an interior space;
a membrane at least partially received within the interior space of the housing and moveable from a first position within the housing to a second position within the housing spaced from the first position;
a needle hub positioned at the first end of the housing and configured to be connected to a syringe;
a needle connected to the needle hub, the needle having a first end and a second end positioned opposite the first end;
a first connection interface defined by a portion of the housing; and
a connection release member moveable relative to the housing between a first position and a second position; and
a secondary component comprising:
a component housing having a first end and a second end positioned opposite the first end, the component housing includes a sidewall defining passageway;
a component membrane at least partially received within the passageway; and
a second connection interface positioned on the component housing, the second connection interface is configured to engage the first connection interface to secure the syringe adapter to the secondary component when the secondary component is at least partially received within the housing of the syringe adapter, wherein the connection release member is configured to engage the second connection interface and release the second connection interface from the first connection interface when the connection release member is moved to the second position.

2. The system of claim 1, wherein a portion of the component housing is configured to be received within the interior space of the housing of the syringe adapter to move the membrane from the first position to the second position, and wherein the first connection interface is configured to engage the second connection interface when the portion of the component housing is positioned within the interior space of the housing of the syringe adapter with the membrane in the second position.

3. The system of claim 1, wherein the syringe adapter further comprises a membrane housing receiving the membrane, and wherein the membrane housing is configured to connect to the component housing when the secondary component is at least partially received within the housing of the syringe adapter.

4. The system of claim 1, wherein the secondary component comprises a luer connector.

5. The system of claim 1, further comprising a biasing member received within the housing, the biasing member biasing the membrane toward the first position.

6. The system of claim 1, wherein the first connection interface comprises a ledge defined by the second end of the housing and the second connection interface comprises a projection.

7. The system of claim 6, wherein the projection of the second connection interface is positioned on a cantilever arm extending from the component housing, and wherein, engagement between the projection and the ledge prevents the secondary component from being removed from the syringe adapter.

8. The system of claim 7, wherein engagement between the projection and the ledge comprises a snap-fit and can only be disengaged via the connection release member.

9. The system of claim 1, wherein the connection release member comprises an arm, and wherein the arm is configured to be pressed by a healthcare to move a portion of the arm radially inward into engagement with the second connection interface.

10. A system for closed transfer of fluids comprising:
a syringe adapter comprising:
a housing having a first end and a second end positioned opposite the first end, the housing includes a sidewall defining an interior space;
a membrane at least partially received within the interior space of the housing and moveable from a first position within the housing to a second position within the housing spaced from the first position;
a needle hub positioned at the first end of the housing and configured to be connected to a syringe; and
a needle connected to the needle hub, the needle having a first end and a second end positioned opposite the first end;
a secondary component comprising:
a component housing having a first end and a second end positioned opposite the first end, the component housing includes a sidewall defining passageway; and
a component membrane at least partially received within the passageway; and
a locking collar connected to and moveable relative to one of the syringe adapter and the secondary component, the locking collar comprising a first threaded portion configured to engage a second threaded portion provided on the other of the syringe adapter and the secondary component when the secondary component is at least partially received within the housing of the syringe adapter.

11. The system of claim 10, wherein the locking collar is connected to the syringe adapter and moveable relative to the syringe adapter in axial and rotational directions, and wherein the secondary component comprising a flange including the second threaded portion.

12. The system of claim 10, wherein the syringe adapter comprises a first connection interface defined by a portion of the housing, and wherein the secondary component comprises a second connection interface positioned on the component housing, the second connection interface is configured to engage the first connection interface to secure the syringe adapter to the secondary component when the secondary component is at least partially received within the housing of the syringe adapter.

13. The system of claim 12, wherein the locking collar has a locked position where the first threaded portion is engaged with the second threaded portion when the first connection interface is engaged with the second connection interface and an unlocked position where the first threaded portion is disengaged from the second threaded portion.

14. The system of claim 10, wherein the syringe adapter further comprises a membrane housing receiving the membrane, and wherein the membrane housing is configured to connect to the component housing when the secondary component is at least partially received within the housing of the syringe adapter.

15. The system of claim 10, wherein the first connection interface comprises a ledge defined by the second end of the housing and the second connection interface comprises a projection.

16. The system of claim 15, wherein the projection of the second connection interface is positioned on a cantilever arm extending from the component housing, and wherein, engagement between the projection and the ledge connects the secondary component to the syringe adapter while allowing from disconnection of the secondary component upon separation of the secondary component from the syringe adapter.

17. The system of claim 10, wherein the locking collar is connected to the secondary component and rotatable relative to the secondary component, and wherein the second threaded portion is positioned on the housing of the syringe adapter.

18. A system for closed transfer of fluids comprising:
a syringe adapter comprising:
a housing having a first end and a second end positioned opposite the first end, the housing includes a sidewall defining an interior space;
a membrane at least partially received within the interior space of the housing and moveable from a first position within the housing to a second position within the housing spaced from the first position;
a needle hub positioned at the first end of the housing and configured to be connected to a syringe; and
a needle connected to the needle hub, the needle having a first end and a second end positioned opposite the first end; and
a secondary component comprising:
a component housing having a first end and a second end positioned opposite the first end, the component housing includes a sidewall defining passageway; and
a component membrane at least partially received within the passageway,
wherein one of the housing of the syringe adapter and the component housing of the secondary component includes a latch arm, and wherein the other of the housing of the syringe adapter and the component housing of the secondary component includes a latch post, the latch arm configured to engage the latch post to secure the syringe adapter to the secondary component when the secondary component is at least partially received within the housing of the syringe adapter.

19. The system of claim 18, wherein the latch arm is positioned on the housing of the syringe adapter and moveable relative to the housing of the syringe adapter, and wherein the latch post extends radially outward from the component housing of the secondary component.

20. A system for closed transfer of fluids comprising:
a syringe adapter comprising:
a housing having a first end and a second end positioned opposite the first end, the housing includes an elliptical sidewall defining an interior space, the elliptical sidewall of the housing having a major axis and a minor axis;
a membrane at least partially received within the interior space of the housing and moveable from a first position within the housing to a second position within the housing spaced from the first position;
a needle hub positioned at the first end of the housing and configured to be connected to a syringe; and
a needle connected to the needle hub, the needle having a first end and a second end positioned opposite the first end; and
a secondary component comprising:
a component housing having a first end and a second end positioned opposite the first end, the component housing includes an elliptical sidewall defining passageway, the elliptical sidewall of the component housing having a major axis and a minor axis; and
a component membrane at least partially received within the passageway,
wherein, with the major axis and the minor axis of the elliptical sidewall of the component housing aligned with the major axis and the minor axis of the elliptical sidewall of the housing, the secondary component is configured to be at least partially inserted into the housing of the syringe adapter, and
wherein, with the secondary component at least partially inserted into the housing of the syringe adapter, the secondary component is configured to be locked to the syringe adapter when the major axis of the elliptical sidewall of the component housing is aligned with the minor axis of the elliptical sidewall of the housing.
